(19) United States
(12) Patent Application Publication
(10) Pub. No.: US 2020/0124598 A1
(43) Pub. Date: Apr. 23, 2020

(54) ELECTROCHEMICAL BIO-MARKER DETECTION METHOD AND DEVICE FOR DIAGNOSING DIABETES

(71) Applicants: NDD, INC., Gumi-si, Gyeongsangbuk-do (KR); GUMI ELECTRONICS & INFORMATION TECHNOLOGY RESEARCH INSTITUTE, Gumi-si, Gyeongsangbuk-do (KR)

(72) Inventors: Hyung Jin KIM, Gimcheon-si, Gyeongsangbuk-do (KR); Su Han LEE, Gumi-si, Gyeongsangbuk-do (KR); Min Ji CHOI, Gyeongsangbuk-do (KR); Sae Young AHN, Gumi-si, Gyeongsangbuk-do (KR); Hyun Hwa KWON, Gumi-si. Gyeongsangbuk-do (KR); Sang Keun SUNG, Daejeon (KR)

(73) Assignees: NDD, INC., Gumi-si Gyeongsangbuk-do (KR); GUMI ELECTRONICS & INFORMATION TECHNOLOGY RESEARCH INSTITUTE, Gumi-si, Gyeongsangbuk-do (KR)

(21) Appl. No.: 16/615,121

(22) PCT Filed: Dec. 20, 2018
(86) PCT No.: PCT/KR2018/016340

§ 371 Date: Nov. 19, 2019

(30) Foreign Application Priority Data
Apr. 27, 2018 (KR) 10-2018-0048909

(51) Int. Cl. G01N 33/543; G01N 27/12; G16H 50/30

(52) U.S. Cl. CPC G01N 33/54386; G16H 50/30; G01N 27/127

(57) ABSTRACT

A diagnostic cartridge includes a chip, in which a channel connecting an inlet and an outlet of the liquid is formed, a biosensor adjacent to the channel, and a circuit for supplying power to the biosensor, and when the target material binds to the biosensor, the resistance of the biosensor may be changed.

### TECHNICAL FIELD

The technical field relates to a method and an apparatus for detecting a target material in a liquid, in particular, a method and an apparatus for detecting a bio-marker in a liquid for the diagnosis of diabetes using a diagnostic cartridge comprising a biosensor.

### BACKGROUND ART

Diabetes is a type of metabolic disorder caused when insulin is insufficiently secreted or normal function is not achieved, and is characterized by hyperglycemia with high blood glucose levels, which causes a variety of symptoms and signs and makes glucose excreted into urine. Diabetes is divided into type 1 and type 2, and type 1 diabetes is caused by the body's inability to produce insulin. Type 2 diabetes, which is relatively deficient in insulin, is caused by insulin resistance. Type 2 diabetes seems to be affected by environmental factors such as high calorie, high fat and high protein diets due to westernization of dietary habits, lack of exercise, and stress, but diabetes may be caused by defects in certain genes as well as by pancreatic surgery, infections, and drugs.

In general, diabetes is diagnosed with a blood test. If there is no symptom, diabetes is determined if the blood glucose level measured after fasting for 8 hours or more is 126 mg/dL or more, or the blood glucose level after 2 hours of oral glucose tolerance test is 200 mg/dL or more. Diabetes is diagnosed even when blood glucose measured regardless of meal is 200 mg/dL or more, while drinking a lot of water or urinating more, and losing weight.

Korean Patent Laid-Open No. 10-2017-0072650 (published on Jun. 27, 2017) discloses a kit for diagnosing diabetes mellitus. The disclosed invention relates to a diagnostic kit for diabetes comprising a quantification device for serum metabolites selected from the group consisting of C16, PC ae C36:0, glycine, lysoPC a C18:2, and any combination thereof. The disclosed invention provides diagnosis of diabetes and information necessary for diagnosis of diabetes using serum metabolites.

### DISCLOSURE

### Technical Problem

An example may provide an apparatus and a method for detecting a bio-marker for the diagnosis of diabetes in a liquid.

In an example, a diagnostic cartridge for detecting a bio-marker for the diagnosis of diabetes comprising a biosensor may be provided.

### Technical Solution

According to one aspect, a diagnostic cartridge for diagnosing diabetes comprises a nano wire-based biosensor formed based on an indium tin oxide microelectrode (ITO).

According to another aspect, a diagnostic cartridge for detecting a target material included in a liquid comprises a top plate, in which a channel connecting an inlet and an outlet of the liquid is formed, a chip coupled to the top plate to form the channel, a biosensor adjacent to the channel, and a circuit for supplying power to the biosensor, and when the target material binds to the biosensor, the resistance of the biosensor is changed.

The nano wire may be formed on a substrate of the biosensor based on an indium tin oxide microelectrode (ITO).

The nano wire may comprise an identification material capable of specifically binding to the target material, wherein the identification material may be fixed to a surface of the indium tin oxide (ITO).

The target material may be an antigen, the identification material may be an antibody, and the specific binding may be an antigen-antibody response.

The antigen may be a preset antigen for diagnosing diabetes.

When the target material and the identification material bind to each other, the resistance of the nano wire may be changed by the binding.

The nano wire is plural.

The diagnostic cartridge may further comprise a liquid separation device for dividing the liquid into a plurality of channels, the top plate may have a plurality of channels, and the channels may be connected to the liquid separation device, respectively.

The outlet may comprise a pad capable of absorbing the liquid.

The biosensor is detachable from the chip.

According to one aspect, a method of detecting a target material included in a liquid comprises the steps of: supplying power to a diagnostic cartridge into which the liquid has been introduced; measuring a current generated by the power; and detecting the target material by determining whether the liquid includes the target material based on the measured current.

The diagnostic cartridge comprises a top plate, in which a channel connecting an inlet and an outlet of the liquid is formed, a chip coupled to the top plate to form the channel, a biosensor adjacent to the channel, and a circuit for supplying power to the biosensor, and when the target material binds to the biosensor, the resistance of the biosensor may be changed.

The biosensor may comprise a nano wire, and a source electrode and a drain electrode, which are positioned at both ends of the nano wire and are connected to the circuit, respectively.

The nano wire may be formed on the substrate of the biosensor based on the indium tin oxide microelectrode (ITO).

The step of measuring the current generated by the power may comprise the steps of: measuring a plurality of currents generated by a plurality of nano wires of the biosensor; and calculating the current based on the plurality of currents.

According to another aspect, an application for detecting a target material is stored in a data processing device and controls the data processing device to perform the steps of: supplying power to a diagnostic cartridge into which a liquid has been introduced; measuring a current generated by the power; and detecting the target material by determining whether the liquid includes the target material based on the measured current.

According to another aspect, an apparatus for detecting a target material included in a liquid comprises a memory in which a program for detecting the target material included in the liquid is recorded, and a processor for executing the program, wherein the program comprises the steps of: supplying power to a diagnostic cartridge into which the liquid has been introduced; measuring a current generated by the power; and detecting the target material by determining whether the liquid includes the target material based on the measured current.

According to another aspect, a method of calculating a risk for a disease based on a target material included in a liquid comprises the steps of: supplying power to a diagnostic cartridge into which the liquid has been introduced; measuring a current generated by the power; detecting the target material by determining whether the liquid includes the target material based on the measured current; and calculating the risk for the disease using the target material and an algorithm.

According to another aspect, an apparatus for calculating a risk for a disease based on a target material included in a liquid comprises a memory in which a program for calculating the risk for the disease is recorded, and a processor for executing the program, wherein the program comprises the steps of: supplying power to a diagnostic cartridge into which the liquid has been introduced; measuring a current generated by the power; detecting the target material by determining whether the liquid includes the target material based on the measured current; and calculating the risk for the disease using the target material and an algorithm.

### Advantageous Effects

An apparatus and a method for detecting a target material in a liquid in a non-invasive manner may be provided.

A diagnostic cartridge for detecting a target material comprising a biosensor may be provided.

### DESCRIPTION OF DRAWINGS

FIGS. 1 and 2 show configuration diagrams of a diagnostic cartridge according to an example.

FIG. 3 shows a biosensor according to an example.

FIG. 4 shows a process of producing a biosensor according to an example.

FIG. 5 shows a process of producing a biosensor according to another example.

FIG. 6 shows a process of producing a biosensor according to another example.

FIG. 7 shows a relationship between voltage and current depending on width of a nano wire according to an example.

FIG. 8 shows resistance and conductivity depending on width of nano wire according to an example.

FIG. 9 shows a method of binding an identification material to a nano wire according to an example and a method of further processing the nano wire.

FIG. 10 shows a method of binding an identification material to a nano wire according to another example and a method of further processing the nano wire.

FIG. 11 shows a process in which a target material is coupled to a identification material of a biosensor according to an example.

FIG. 12 shows the resistance change rate of the nano wire depending on the concentration of a target material before and after oxygen-plasma treatment according to an example.

FIG. 13 shows a method of detecting a target material using an additional identification material according to an example.

FIG. 14 shows a liquid separation device according to an example.

FIG. 15 shows a diagnostic cartridge comprising a liquid separation device according to an example.

FIG. 16 shows a configuration diagram of a diagnostic cartridge comprising a liquid separation device according to another example.

FIG. 17 shows a configuration diagram of a diagnostic system according to an example.

FIG. 18 shows a configuration diagram of a diagnostic system according to another example.

FIG. 19 shows a flowchart of a method of detecting a target material in a liquid according to an example.

FIG. 20 shows a flowchart of a method of measuring a current generated by a power according to an example.

FIG. 21 shows a flowchart of a method of outputting the risk according to an example.

FIG. 22 shows a flowchart of a method of adjusting an algorithm for calculating the risk according to an example.

FIG. 23 shows a method of controlling a medical device based on the risk according to an example.

### BEST MODE

Hereinafter, the examples will be described in more detail with reference to the appended drawings. However, the scope of the patent application is not limited or limited by these examples. The same reference numerals provided in each drawing denote same members.

Various modifications may be made to the examples described below. The examples described below are not intended to be limited to the embodiments and should be understood to include all modifications, equivalents, and substitutes for them.

The terms used in examples are for the purpose of describing particular example only and are not intended to limit examples. Singular expressions include plural expressions unless the context clearly indicates otherwise. In this specification, terms such as “comprise” and “have” are intended to indicate that there is a feature, number, step, operation, component, part, or combination thereof described in the specification, and it should be understood that the terms do not exclude in advance the possibility of the presence or addition of one or more other features, numbers, steps, operations, components, parts, or combinations thereof.

Unless defined otherwise, all terms used herein, including technical or scientific terms, have the same meaning as commonly understood by those of ordinary skill in the art to which an example belongs. Terms such as those defined in the commonly used dictionaries should be construed as having meanings consistent with the meanings in the context of the related art, and are not be construed in ideal or excessively formal meanings unless expressly defined in this application unless clearly defined in the present application.

In addition, in the description with reference to the appended drawings, the same components will be given the same reference numerals regardless of the reference numerals and duplicate description thereof will be omitted. In describing the examples, if it is determined that the specific description of the related known technologies may unnecessarily obscure the gist of the example, the detailed description will be omitted.

A biosensor is a biosensor capable of detecting a specific material by using a biosensing material that can selectively react with and bind to the specific material in order to confirm whether a specific biomaterial such as a protein, a gene, a hormone, or a virus is present. Biosensors may be usefully used in various fields, such as in the fields of food safety investigation and environmental monitoring as well as in the field of medical diagnostics.

Nano-sized materials have recently emerged as a very important research area because they have new physicochemical properties such as unique electrical, optical and mechanical properties. In particular, the nano-sized elements may be used in various kinds of sensors since they are small in size and have a high surface area/volume ratio, and thus making electrochemical reactions occurring on the surface of the elements active.

According to one aspect, a diagnostic cartridge comprising a biosensor may be used to detect a specific material included in a liquid (for example, blood, saliva, or urine). The specific material to be detected is called a target material. The target material may be used as a bio-marker of certain diseases. A method of diagnosing diabetes through blood is an invasive inspection method, and a method of diagnosing diabetes through urine or saliva is a noninvasive inspection method.

When the user collects saliva and inserts it into the diagnostic cartridge, the target material specifically binds to each other with the biosensors in the diagnostic cartridge. The binding may change the electrical properties of the biosensor. The user may detect the target material in saliva by measuring the changed electrical properties. Further, the user may also calculate the concentration of the target material based on the amount of change in the electrical property.

For example, the target material may be a preset material for diagnosing diabetes. The target material may be a causative material causing diabetes or a material caused by diabetes, and may be an antigen.

A material that can specifically bind to the target material may be included in the biosensor. The material included in the biosensor is defined as an identification material and may be an antibody.

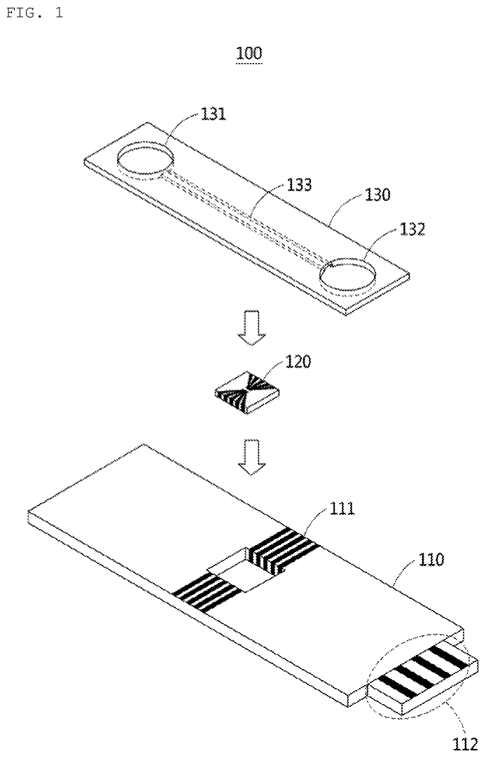

Hereinafter, a method and a diagnostic cartridge for detecting a target material will be described in detail with reference to FIGS. 1 to 24.

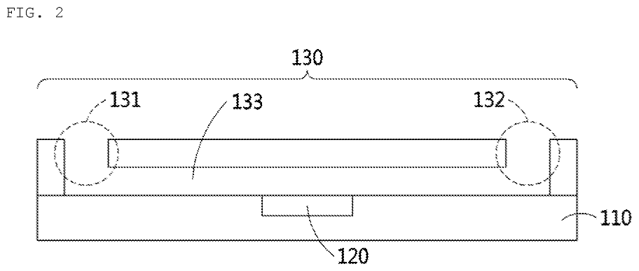

FIGS. 1 and 2 show configuration diagrams of a diagnostic cartridge according to an example.

According to an aspect, the diagnostic cartridge 100 comprises a chip 110, a biosensor 120, and a top plate 130.

The chip 110 comprises circuits 111 and 112 for supplying power to the biosensor 120. Although the circuit 111 is shown to appear outside the chip 110, the circuit 111 may be formed inside the chip 110. The circuit 111 and the circuit 112 are connected to each other, and the circuit 112 may receive power from an external device. For example, the circuit 112 may be embodied in the form of a universal serial bus (USB) or a connector of a smartphone. The chip 110 may be manufactured using a material such as glass or plastic.

The top plate 130 may comprise a channel 133 connecting an inlet 131 and an outlet 132 of a liquid (for example, saliva). The channel 133 may be formed below the top plate 130. When the top plate 130 and the chip 110 are coupled to each other, the channel 133 may be formed between the top plate 130 and the chip 110. The liquid infused through the inlet 131 may pass through the biosensor 120 through the channel 133, and the passed liquid passing through the biosensor 120 may be discharged to the outlet 132. The top plate 130 may be made of polydimethylsiloane (PDMS) or plastic. The top plate 130 may be manufactured in an injection type.

The biosensor 120 is adjacent to the channel 133. The structure of the channel 133 and the biosensor 120 may be designed so that the liquid flowing through the channel 133 may be in direct contact with the biosensor 120. The biosensor 120 maybe physically separated from the chip 110. When the chip 110 and the top plate 130 are coupled to each other, the liquid flows only through the channel 133 and does not leak between the chip 110 and the top plate 130. In addition, the liquid does not leak between the biosensor 120 and the chip 110.

The surface of the channel 133 maybe coated with a hydrophilic material to allow the liquid to flow well. The width of the channel 133 may be 500 micrometer (μm) or more.

The outlet 132 may comprise a hygroscopic pad capable of absorbing liquid. When the liquid is absorbed through the hygroscopic pad, the liquid does not stay in the channel 133 but moves through the channel 133.

FIG. 2 shows a cross section of a diagnostic cartridge 100. A channel 133 is formed on a chip 110 and a biosensor 120, and a liquid flows from an inlet 131 to an outlet 132 through a channel 133.

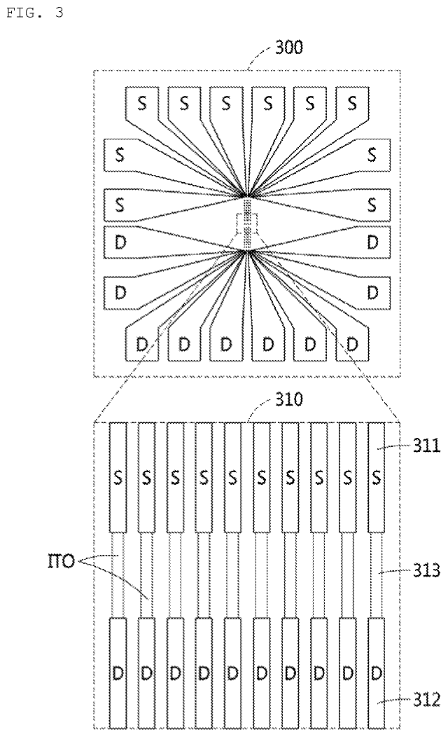

Hereinafter, the structure and the production process of the biosensor 120 will be described in detail with reference to FIGS. 3 to 13.

FIG. 3 shows a biosensor according to an example.

According to one aspect, the biosensor 300 maybe a transistor. For example, the biosensor 300 may be the biosensor 120 as described above with reference to FIGS. 1 and 2. The biosensor 300 comprises a circuit comprising a gate electrode, a source electrode, and a drain electrode. The biosensor 300 comprises a plurality of source electrodes and a plurality of drain electrodes. Each of the plurality of source electrodes and the plurality of drain electrodes is electrically connected to each other. For example, the source electrode and the drain electrode are connected through a nano wire. A nano wire is a nanoscale connection line. The biosensor 300 may further comprise a nano thin film. For example, the nano thin film and the nano wire may be formed of an indium tin oxide microelectrode (ITO).

When the biosensor 300 is coupled to the chip 110, the source electrode and the drain electrode of the biosensor 300 maybe connected to the circuit 111 of the chip 110, respectively.

The enlarged view 310 shows the connection relationship between the source electrode 311, the nano wire 313, and the drain electrode 312 in detail. When the target material is bonded to the nano wire 313, the resistance value of the nano wire 313 is changed by the target material. As the resistance value is changed, the magnitude of the current flowing between the source electrode 311 and the drain electrode 312 is changed. The target material may be detected based on the changed magnitude of the current.

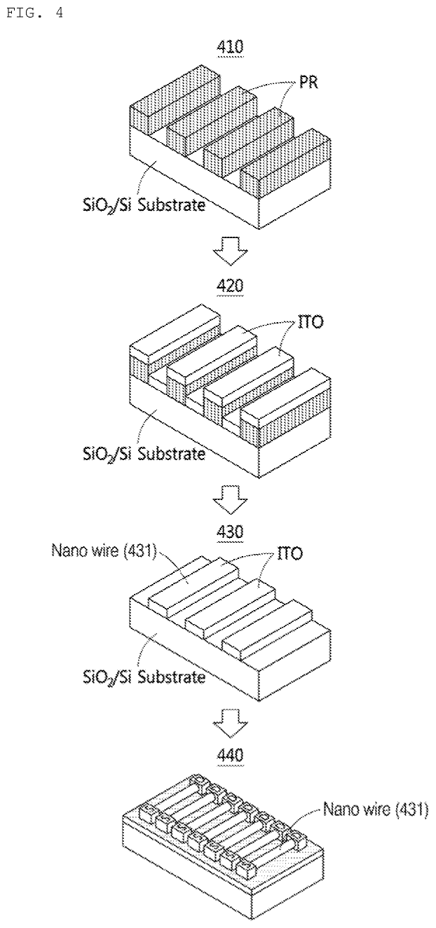

Referring to FIG. 4 below, a process of producing the nano wire 313 in the biosensor 300 is showed.

FIG. 4 shows a process of producing a biosensor according to an example.

Step 410 shows the step of forming a photoresist layer. A photoresist pattern is formed on the substrate. The method of forming the photoresist pattern may be UV lithography, X-ray lithography, electron beam lithography, ion beam lithography, or the like.

The width of the photoresist pattern may be controlled in a nano unit. For example, the width of the photoresist pattern is controlled in a nano unit by using a plasma downstream ashing process. The line width of the photoresist pattern is adjusted in nano units of 1 nm to 10 nm. Ashing using plasma is a method in which a photoresist pattern is etched using a plasma generator, and the line width of the photoresist pattern becomes narrower as it is exposed to the plasma generator for a long time.

The substrate may be a silicon (Si) wafer and a wafer deposited with silicon oxide (SiO₂), a glass substrate and a glass substrate coated with a transparent conductive oxide film, a flexible organic substrate such as a polymer, and a metal.

Step 420 shows the step of applying an indium tin oxide (ITO) on the substrate on which the photoresist pattern is formed. For example, ITO is applied by the thickness of a nano wire to be produced.

Step 430 shows the step of removing the photoresist pattern formed on the substrate. When the photoresist pattern is removed, the ITO applied on the photoresist pattern is also removed together. The pattern of ITO remaining on the substrate forms a nano wire 431.

Step 440 shows the step of producing a source electrode in electrical contact with the nano wire 431 and a drain electrode spaced apart from the source electrode. When a voltage is applied to each of the source electrode and the drain electrode, current may flow through the nano wires 431. The nano wire 431 has respective resistance values according to the thickness, length, and width of the nano wire 431.

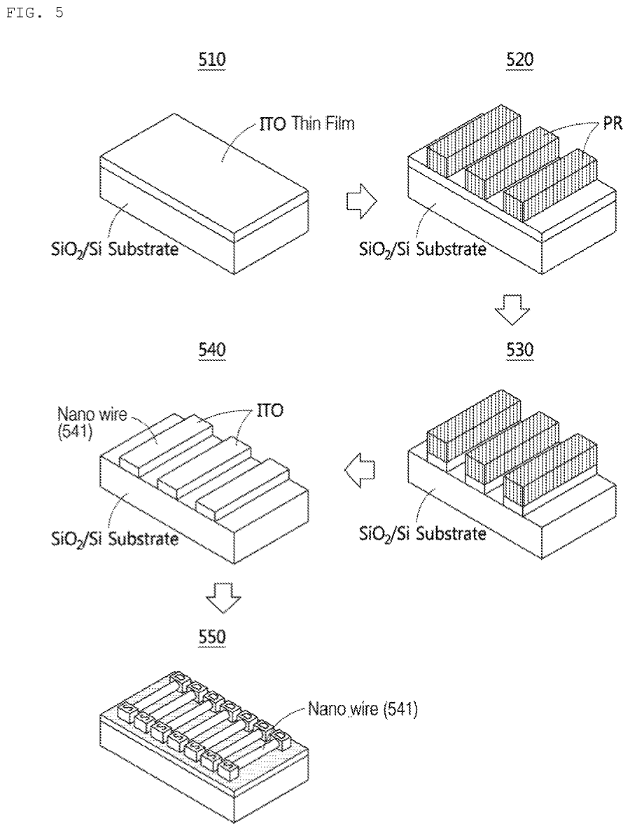

FIG. 5 shows a process of producing a biosensor according to another example.

Step 510 shows the step of applying an ITO thin film on the substrate. For example, an ITO thin film is applied by the thickness of the nano wire to be produced.

Step 520 shows the step of forming a photoresist layer on the substrate to which the ITO thin film is applied. For example, a photoresist pattern is formed on the substrate by the width of the nano wire to be produced.

Step 530 shows the step of removing the ITO thin film applied to the portion on which the photoresist pattern is not formed.

Step 540 shows the step of removing the photoresist pattern. When the photoresist pattern is removed, the pattern of ITO remaining on the substrate forms a nano wire 541.

Step 550 shows the step of producing a source electrode in electrical contact with the nano wire 541 and a drain electrode spaced apart from the source electrode.

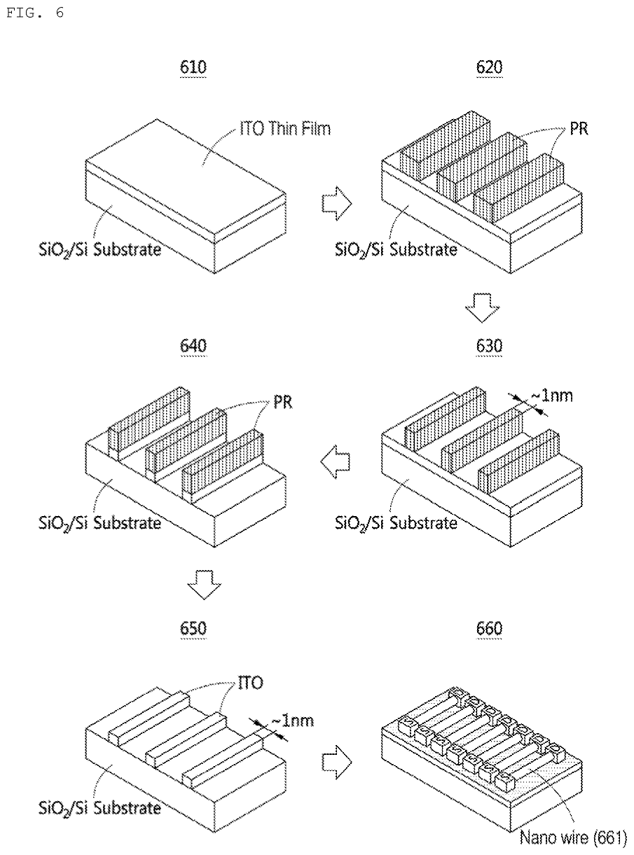

FIG. 6 shows a process of producing a biosensor according to another example.

Step 610 shows the step of applying an ITO thin film on the substrate. For example, an ITO thin film is applied by the thickness of the nano wire to be produced.

Step 620 shows the step of forming a photoresist pattern on the substrate to which the ITO thin film is applied. The thickness of the photoresist pattern may be 5 nm or less.

Step 630 shows the step of adjusting the width of the photoresist pattern to correspond to the width of the nano wire to be produced. A portion of the photoresist pattern may be removed through an ashing process. For example, the width of the nano wire to be produced may be 1 nm or less.

Step 640 shows the step of removing the ITO thin film applied to the portion on which the photoresist pattern is not formed. A portion of the ITO thin film may be removed through an etching process.

Step 650 shows the step of removing the photoresist pattern. When the photoresist pattern is removed, the pattern of ITO remaining on the substrate forms a nano wire 661.

Step 660 shows the step of producing a source electrode in electrical contact with the nano wire 661 and a drain electrode spaced apart from the source electrode.

FIG. 7 shows a relationship between voltage and current depending on width of a nano wire according to an example.

The wider the line width of the nano wire, the higher the current generated for the same voltage. The width of the nano wire may be appropriately determined depending on the target material or bio-marker used to diagnose a specific disease.

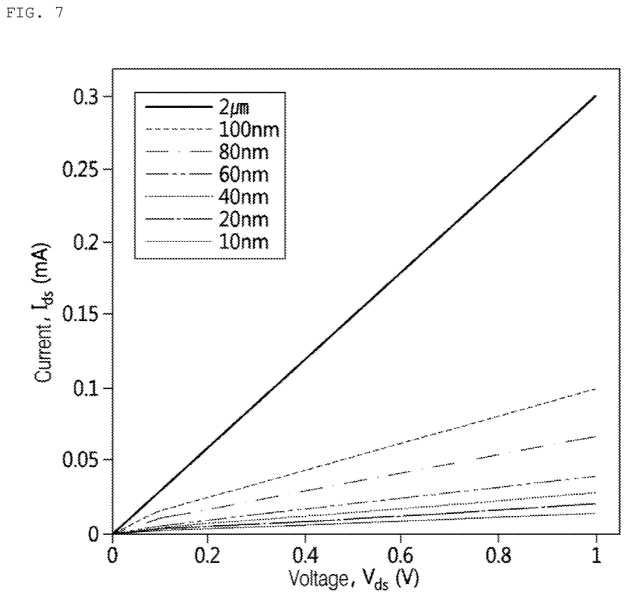

FIG. 8 shows resistance and conductance depending on width of nano wire according to an example.

As the width of the nano wire increases, the resistance of the nano wire decreases and the conductance having a reciprocal relationship with resistance increases.

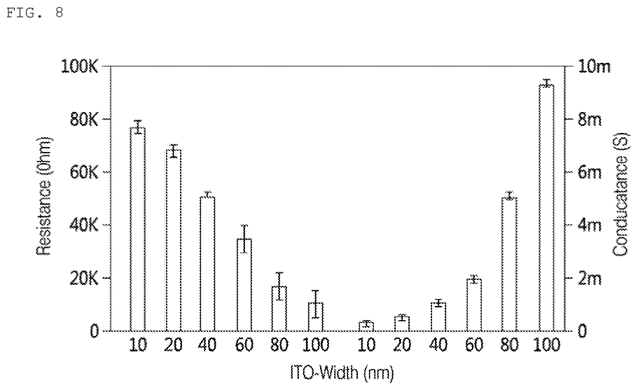

FIG. 9 shows a method of binding an identification material to a nano wire according to an example and a method of further processing the nano wire.

In order to detect a target material in a liquid, an identification material, which is a material capable of specifically binding to the target material, is used. Specific binding may be an antigen-antibody response. The identification material may be a protein, such as an antigen, an antibody, an enzyme, a peptide, and a polypeptide, for diagnosing and preventing a disease. As the identification material may use a peptide nucleic acid (PNA), a locked nucleic acid (LNA), and RNA, as well as a protein, as a detection receptor. The target material is not limited as long as it specifically binds to and reacts with the identification material, and the target material may include a protein such as an antigen, an antibody, an enzyme, a peptide, and a polypeptide. The target material is not limited to the described examples as long as it specifically binds to an identification material other than a protein, and the target material may include PNA, LNA, RNA, DNA, bacteria, viruses, and the like.

According to one aspect, glycated albumin (GA) may be used as a target material for diagnosing diabetes, and glucose may be used as an identification material capable of specifically binding to the target material. In addition, the target material includes monomers and oligomers produced based on glycated albumin. The target material may be detected from blood other than saliva or urine. The accuracy of diabetic diagnosis may be further improved by comparing and analyzing the factors detected from blood and those detected from saliva (or urine).

The identification material is fixed (or bonded) to the surface of the nano wire, and when the identification material binds to the target material, the resistance value of the nano wire may be changed.

The nano wire 911 in step 910 is coupled with an intermediate material 912 for attaching the identification material 913. The identification material 913 is attached to the nano wire 911 by physically or chemically binding to the intermediate material 912.

Although the nano wire 911 in step 920 may be used to detect the target material, further processing may be performed on the nano wire 911 to efficiently bind to the target material. A blocking layer may be formed on the nano wire 911 to prevent the target material from chemically or physically binding to a material other than the identification material. For example, the nano wire 911 may be immersed in a liquid containing bovine serum albumin (BSA) 922 to form a blocking layer. The BSA 922 may bind to the surface of the nano wire 911 and the functional group of the intermediate material 912 except for the identification material.

The nano line 911 in step 930 may be used to detect the target material.

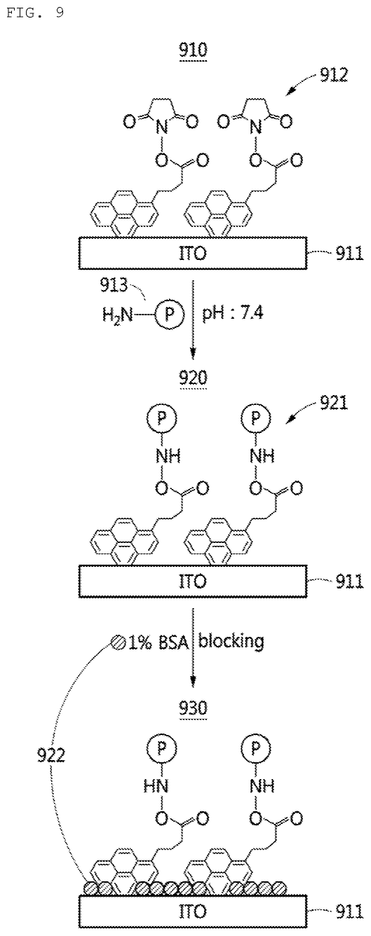

FIG. 10 shows a method of binding an identification material to a nano wire according to another example and a method of further processing the nano wire.

FIG. 9 shows an example in which the intermediate material 912 is first bonded to the nano wire 911, and FIG. 10 shows an example in which the intermediate material 1011 is first bonded with the target material 1012.

In step 1010, the intermediate material 1011 and the target material 1012 may be coupled to each other.

In operation 1020, the intermediate material 1011 bound to the target material 1012 may be coupled to the nano wire 1031.

In step 1030, a blocking layer may be formed on the nano wire 1031 to prevent the target material 1012 from chemically or physically binding to a material other than the identification material. For example, the nano wire 1031 may be immersed in a liquid containing ethanolamine 1032 to form a blocking layer. The ethanolamine 1032 may bind to the surface of the nano wire 1031 and the functional group of the intermediate material 1021 except for the identification material.

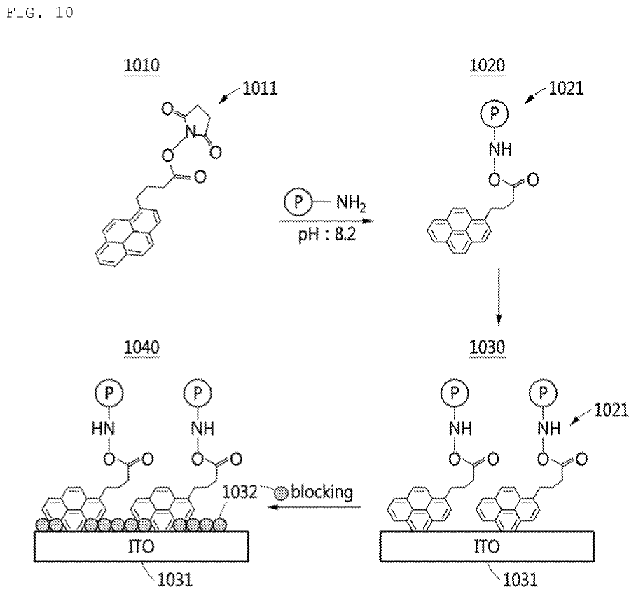

FIG. 11 shows a process in which a target material is coupled to a identification material of a biosensor according to an example.

Step 1110 shows a state in which the nano wire 1111 finally bound to the identification material 1112 has not been coupled to the target material in the liquid. That is, step 1110 shows a state in which the liquid has not been introduced into the biosensor 300.

Step 1120 shows a state in which the target material 1121 has been coupled to the identification material 1112. When the liquid passes through the biosensor 300, the target material 1121 in the liquid is coupled to the identification material 1112.

FIG. 12 shows the resistance change rate of oxygen-plasma-treated ITO nano wires and untreated nano wires according to an example.

The results of detecting the target material using the biosensor produced by the oxygen-plasma treatment on the surface of the ITO nano wire and the biosensor produced by the untreated ITO nano wire are shown.

Oxygen-plasma treatment on the surface of the ITO nano wire allows more identification material to bind to the same area.

When the target material binds to the biosensor, the resistance of the nano wire may be changed. It was measured that the resistance change rate of the oxygen-plasma-treated ITO nano wires was high in all concentration ranges of the target material. In addition, it can be seen experimentally that the resistance change rate of the oxygen-plasma-treated ITO nano wires is high in a high concentration range of the target material.

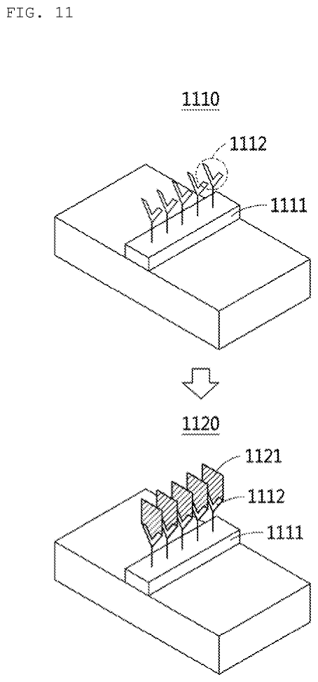

FIG. 13 shows a method of detecting a target material using an additional identification material according to an example.

An additional identification material 1331 or an additional identification material group maybe used so that the target material can be detected more effectively. The additional identification material 1331 or the additional identification material group may be nanoparticles. For example, the additional identification material 1331 may be the same material as the identification material 1112. As another example, the additional identification material 1331 may be a different material from the identification material, which may bind to the target material. Additional identification material 1331 maybe used when the target material may bind to at least two materials simultaneously.

Step 1310 shows the step in which a liquid has been introduced yet. The additional identification material 1331 may be present in the biosensor without being bound to the identification material 1112. For example, when the liquid such as saliva flows through the biosensor, additional identification material 1331 may be arranged to enter the liquid.

Step 1320 shows the step of injecting the liquid into the biosensor to bind the target material 1121 to the identification material 1112. When the target material 1121 binds to the identification material 1112, the resistance of the nano wires 1131 may be changed.

Step 1330 shows the step of further coupling additional identification material 1331 to the target material 1121 bound to the identification material 1112. Since the additional identification material 1331 is further coupled to the nano wire 1111, the resistance of the nano wire 1111 may be further changed.

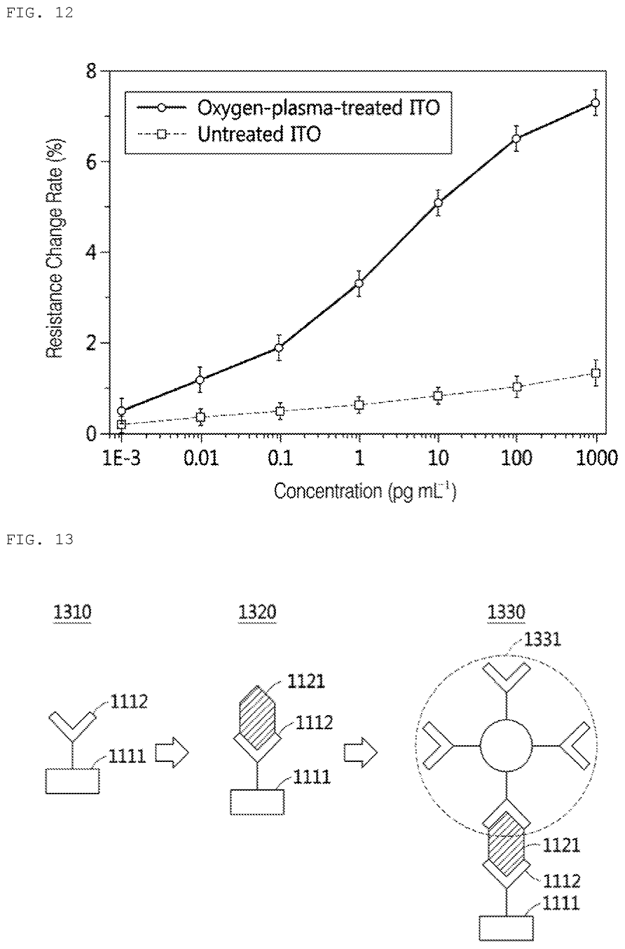

FIG. 14 shows a liquid separation device according to an example.

According to one aspect, the liquid separation device 1400 comprises an inlet 1410, a separation part 1420, a filter part 1430, a first outlet 1440, and a second outlet 1450. For example, the liquid separation device 1400 may be manufactured in the form of an independent device. As another example, the liquid separation device 1400 may be manufactured to be included in the top plate 130 as described above with reference to FIGS. 1 and 2. That is, the liquid separation device 1400 may be formed in the top plate 130.

Before the liquid is supplied to the biosensor 120 as described above with reference to FIGS. 1 and 2, the liquid separation device 1400 may pre-process the liquid.

The inlet 1410 is connected to the inlet 131 of the top plate 130. The liquid (for example, saliva or urine) include various sizes of materials, which may be entangled with each other. When the liquid passes through the separation part 1420, the entangled materials may be separated from each other. For example, the entangled materials maybe separated from each other due to centrifugal force, centripetal force, weight difference, and size difference.

Although the passage of the separation part 1420 is illustrated as a line, the cross section of the passage may be in the form of a trapezoid. For example, the separation part 1420 may be higher in outer height than the inner height of the passage. According to the hydrokinetics, after the liquid passes through the separation part 1420, in the vicinity of the filter part 1430 a large-sized material may be gathered into the inside of the passage and a small-sized material may be gathered in the outside of the passage.

The filter part 1430 may divide the separated materials into a plurality of pathways according to sizes. For example, the filter part 1430 may include a plurality of filters having respective sizes. For example, the filter part 1430 may separate the liquid into a first liquid and a second liquid.

The separated first liquid may proceed to the first outlet 1440. The first outlet 1440 may be connected to the channel 133, and the first liquid may pass through the biosensor 120. The biosensor 120 may bind to a target material among materials included in the first liquid.

The separated second liquid may proceed to the second outlet 1450. For example, the second outlet 1450 may be connected to the outlet 1432. That is, the separated second liquid may be discharged without being treated. As another example, the second outlet 1450 may be connected to a second channel, which is different from the channel 133. The second biosensor may be connected to the second channel. The second biosensor may be a sensor for detecting the second target material in the second liquid.

Although the liquid separation device 1400 is shown to comprise a first outlet 1440 and a second outlet 1450, it may filter the liquid in a plurality of pathways according to the purpose and discharge the separated liquid into a plurality of outlets.

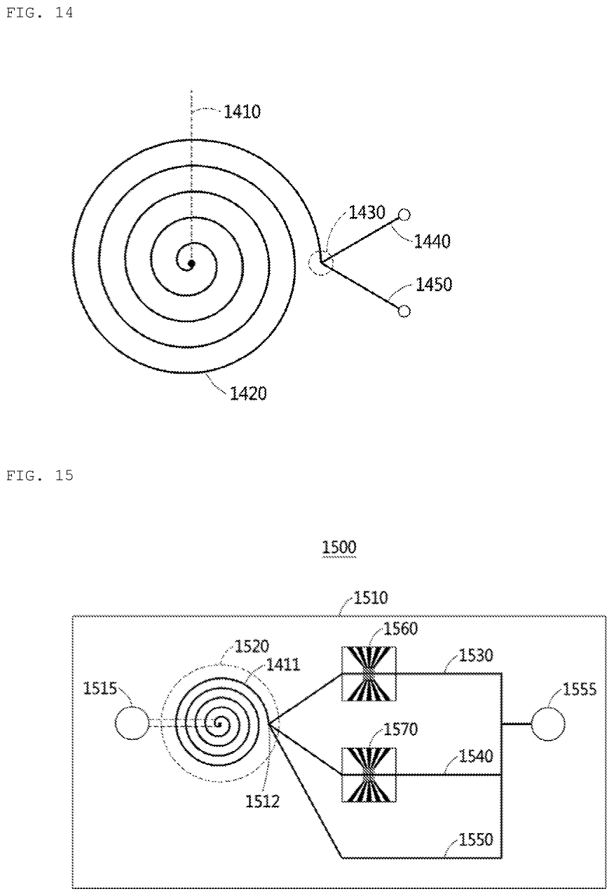

FIG. 15 shows a diagnostic cartridge comprising a liquid separation device according to an example.

The user introduces a liquid into the inlet 1515 of the diagnostic cartridge 1500. The liquid proceeds to a liquid separation device 1520 connected to the inlet 1515. Entangled liquids are separated from each other through the separation part 1520, and the separated liquids may be divided into a plurality of channels 1530, 1540, and 1550 through the filter part 1512.

The first liquid proceeding through the first channel 1530 passes through the first biosensor 1560, and the first biosensor 1560 couples with the first target material included in the first liquid.

The second liquid proceeding through the second channel 1540 passes through the second biosensor 1570, and the second biosensor 1570 couples with the second target material included in the second liquid. For example, the first target material and the second target material may be materials for detecting the same disease. As another example, the first target material and the second target material may be materials for detecting different diseases.

The third liquid proceeding through the third channel 1550 proceeds to the outlet 1555.

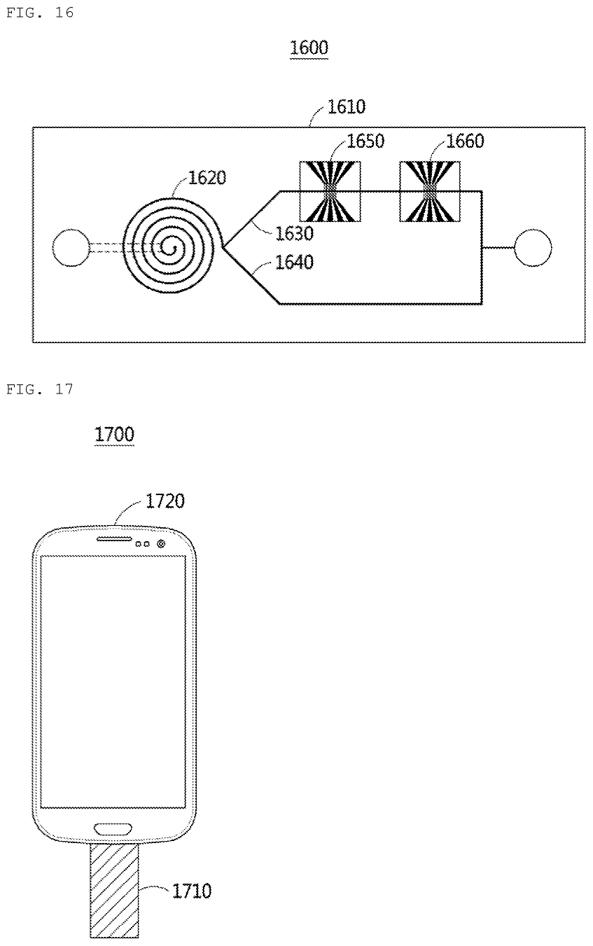

FIG. 16 shows a configuration diagram of a diagnostic cartridge comprising a liquid separation device according to another example.

The user introduces a liquid into the inlet of the diagnostic cartridge 1600. The liquid proceeds to a liquid separation device 1620 connected to the inlet. Entangled liquids are separated from each other through the separation part 1620, and the separated liquids may be divided into a plurality of channels 1630 and 1640 through the filter part.

The first liquid proceeding through the first channel 1630 passes through the first biosensor 1650 and the second biosensor 1660 in turn, the first biosensor 1650 couples with the first target material included in the first liquid, and the second biosensor 1660 couples with the second target material included in the first liquid.

The second liquid proceeding through the second channel 1640 proceeds to the outlet.

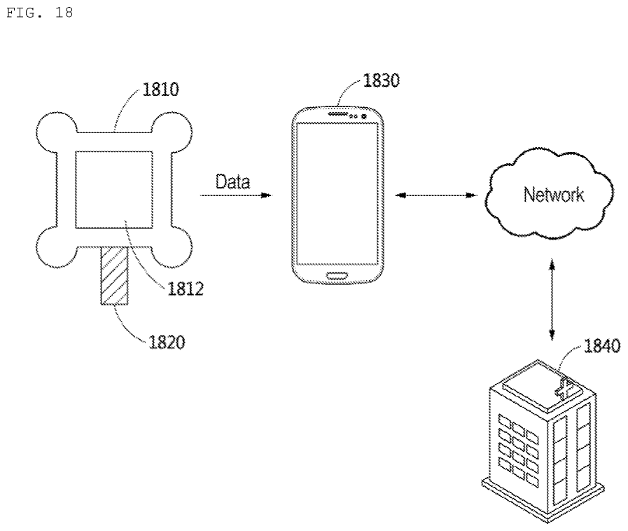

FIG. 17 shows a configuration diagram of a diagnostic system according to an example.

A diagnostic system 1700 comprises a diagnostic device 1720 and diagnostic cartridge 1710. For example, the diagnostic cartridge 1710 may be the diagnostic cartridges 100, 1500, 1600 as described above with reference to FIGS. 1, 2, 15, and 16.

The user may connect the diagnostic cartridge 1710 including the liquid to the diagnostic device 1720. The user may connect the diagnostic cartridge 1710 to the diagnostic device 1720 using a connector of the diagnostic cartridge 1710. The diagnostic device 1720 may supply a voltage to the diagnostic cartridge 1710 through the connector.

According to one aspect, the diagnostic device 1720 may be a dedicated device for detecting the target material in the liquid using the diagnostic cartridge 1710. The diagnostic device 1720 may be an apparatus for detecting a target material.

According to another aspect, the diagnostic device 1720 may be a data processing device that is used universally, and the data processing device may detect a target material in the liquid using an application stored in the device. The data processing device may be a mobile device. For example, the application stored in the mobile processing device may be a application for detecting a target material.

The diagnostic device 1720 includes a communication part, a processor, and a memory.

The communication part is connected to the processor and the memory to transmit and receive data. The communication part may be connected to another external device to transmit and receive data. Hereinafter, the expression transmitting and receiving “A” may refer to transmitting and receiving “information or data indicating A.”

The communication part may be embodied in a circuitry within the diagnostic device 1720. For example, the communication part may include an internal bus and an external bus. As another example, the communication part maybe an element connecting the diagnostic device 1720 and an external device. The communication part maybe an interface. The communication part may receive data from the external device and transmit data to the processor and the memory.

The processor processes data received from the communication part and data stored in the memory. A “processor” may be a data processing device that is embodied in a hardware including a circuit having a physical structure for executing desired operations. For example, desired operations may include code or instructions included in a program. For example, the data processing device embodied in the hardware may include a microprocessor, a central processing unit, a processor core, a multi-core processor, a multiprocessor, an application-specific integrated circuit (ASIC), and a field programmable gate array (FPGA).

The processor executes computer readable codes (for example, software) stored in memory and instructions induced by the processor.

The memory stores data received by the communication part and data processed by the processor. For example, the memory may store a program. The stored program may be a collection of syntaxes that can be coded to detect a target material in the liquid and executed by the processor.

According to one aspect, the memory may include one or more volatile memories, nonvolatile memories, random access memories (RAMs), flash memories, hard disk drives, and optical disk drives.

The memory stores a set of commends (for example, software) for operating the diagnostic device 1720. A set of commands for operating the diagnostic device 1720 is executed by a processor.

The communication part, processor, and memory will be described in detail below with reference to FIGS. 18 to 23.

FIG. 18 shows a configuration diagram of a diagnostic system according to another example.

A diagnostic system includes a diagnostic device 1810 and diagnostic cartridge 1820. For example, the diagnostic cartridge 1810 may be the diagnostic cartridges 100, 1500, 1600 as described above with reference to FIGS. 1, 2, 15, and 16. The user may connect the diagnostic cartridge 1820 including the liquid to the diagnostic device 1810. The user may connect the diagnostic cartridge 1820 to the diagnostic device 1810 using a connector of the diagnostic cartridge 1820. The diagnostic device 1810 may supply a voltage to the diagnostic cartridge 1820 through the connector.

According to one aspect, the diagnostic device 1810 may be a dedicated device for detecting the target material in the liquid using the diagnostic cartridge 1820. The diagnostic device 1810 may output the detected result through the display 1812 so that a user may know. The diagnostic device 1810 may transmit the detected result to the user terminal 1830. For example, the diagnostic device 1810 and the user terminal 1830 may exchange data using short-range wireless communication.

The user terminal 1830 may access an external server 1840 using cellular communication. For example, the external server 1840 may be a data server or a hospital server. The server 1840 may store data. For example, the server 1840 may store a plurality of data for each user. The server 1840 may update data on the corresponding disease by using the received data. The server 1840 may have past and present liquid diagnostic information about patients having the corresponding disease, and may have data matching the liquid diagnostic information and actual symptoms of the patient. The server 1840 may predict disease progression for the liquid diagnostic information of the corresponding patient using the data of other patients. The server 1840 may transmit the predicted disease progression to the user terminal 1830.

The user terminal 1830 may reflect the disease progression received from the server 1840 to modify the diagnostic algorithm installed in the user terminal 1830.

FIG. 19 shows a flowchart of a method of detecting a target material in a liquid according to an example.

The steps 1910 to 1930 below may be performed by the diagnostic devices 1720 and 1810 as described above with reference to FIGS. 17 and 18.

In step 1910, the processor supplies power to the diagnostic cartridges 1710 and 1820 into which the liquid has been introduced. For example, the processor may supply power to diagnostic cartridges 1710 and 1820 through a connector. Power maybe supplied to the source electrodes of the diagnostic cartridges 1710 and 1820 through the power.

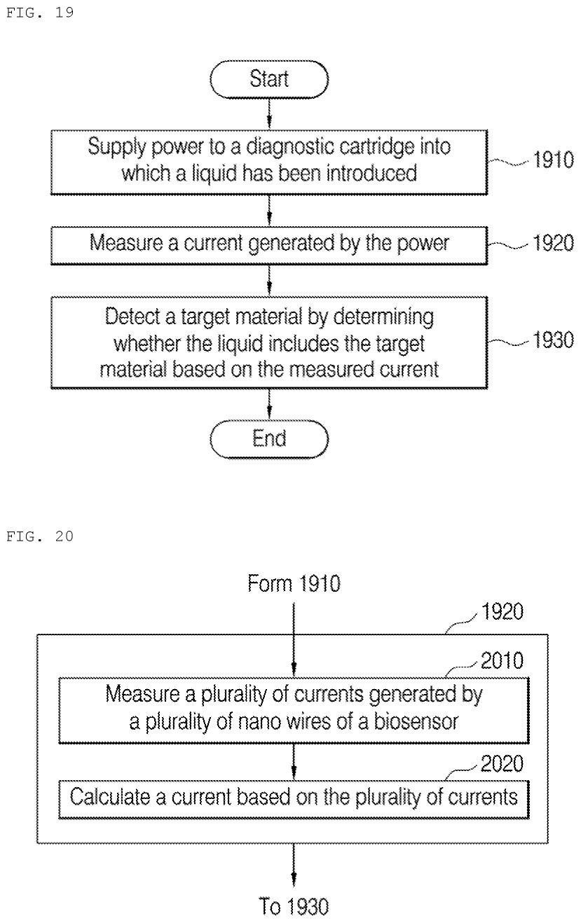

In step 1920, the processor measures the current generated by the power. When power is supplied to the source electrode, current may flow between the source electrode and the drain electrode through the nano wire. The method of measuring the current will be described in detail with reference to FIG. 20 below.

In step 1930, the processor detects the target material by determining whether the liquid includes the target material based on the measured current. For example, if the measured current value corresponds to a preset current value, the target material is detected. As another example, the concentration of the target material may be determined according to the measured current.

FIG. 20 shows a flowchart of a method of measuring a current generated by a power according to an example.

Step 1920 as described above with reference to FIG. 19 includes the steps 2010 and 2020 below.

In step 2010, the processor measures a plurality of currents generated by a plurality of nano wires of the biosensor. The biosensor may include a plurality of source electrodes and a plurality of drain electrodes, and may include nano wires connecting the source electrode and the drain electrode, respectively. The processor may measure the current value flowing through each nano wire.

Instep 2020, the processor may calculate a final current value based on the plurality of current values. For example, the average of the plurality of current values may be calculated as the final current value. As another example, the final current value may be calculated using the remaining current values except at least one of the plurality of current values.

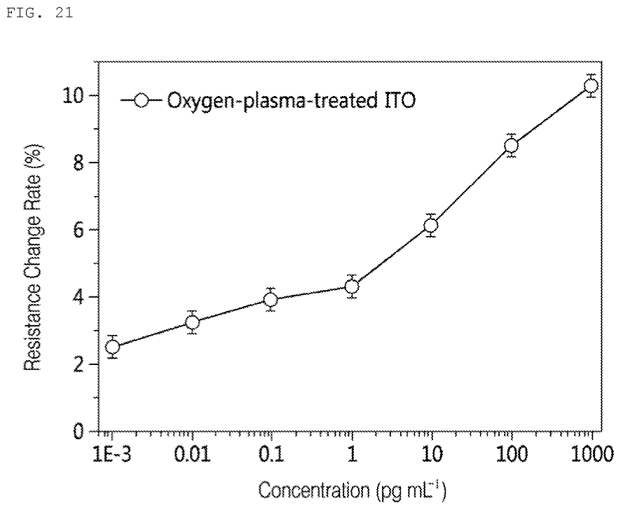

FIG. 21 shows the resistance change rate of the nano wire depending on the concentration of a target material according to an example.

As the concentration of the target material included in the liquid increases, it may be confirmed through experiments that the resistance change rate of the nano wire increases.

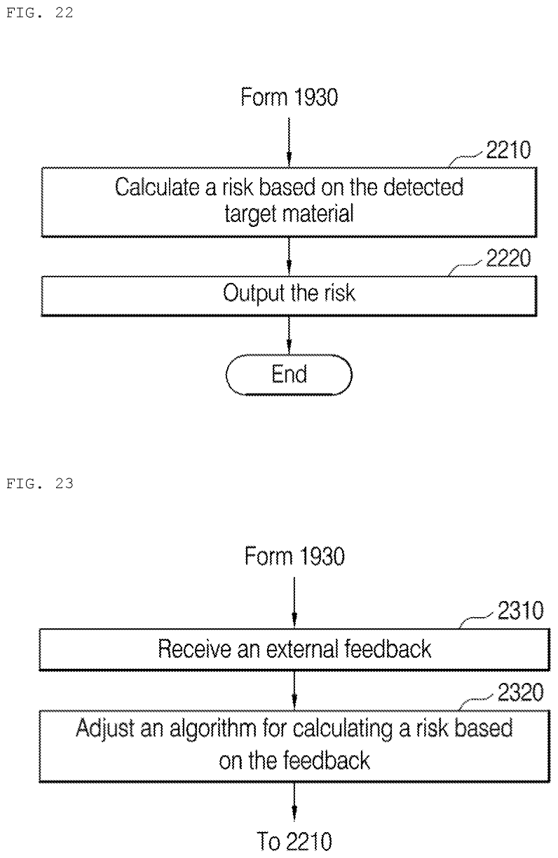

FIG. 22 shows a flowchart of a method of outputting the risk according to an example.

The steps 2210 and 2220 below may be performed by the diagnostic devices 1720 and 1810 as described above with reference to FIGS. 17 and 18. After step 1930 as described above with reference to FIG. 19 is performed, steps 2210 and 2220 may be performed.

In step 2210, the processor of the diagnostic device calculates a risk based on the detected target material. For example, the diagnostic device may calculate the risk based on the concentration of the target material. The higher the concentration of the target material in the liquid, the larger the resistance value of the nano wire, and thus the smaller the current flowing through the nano wire.

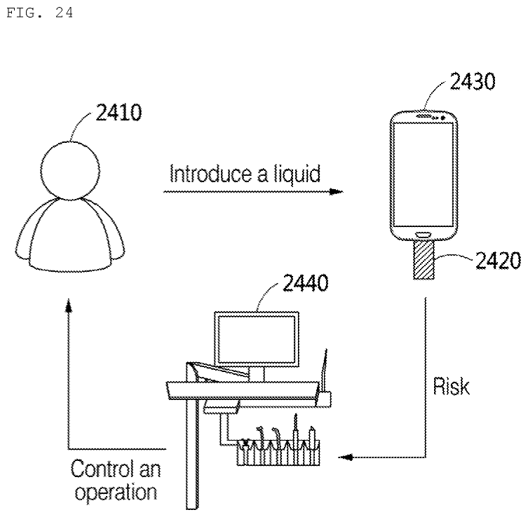

The diagnostic device may calculate the risk for the target material using an algorithm. The algorithm may be stored in advance in the diagnostic device and adjusted or trained for the user. The method of adjusting the algorithm will be described in detail with reference to FIG. 23 below.

When the diagnostic device calculates the risk of a disease using an algorithm, the diagnostic device may be named a device to calculate the risk of onset.

In step 2220, the processor outputs the risk. For example, the processor may generate a risk history for the user and output the history using the display of the diagnostic device. As another example, the processor may transmit the calculated risk to an external server to output it.

FIG. 23 shows a flowchart of a method of adjusting an algorithm for calculating the risk according to an example.

The steps 2310 and 2320 below may be performed by the diagnostic devices 1720 and 1810 as described above with reference to FIGS. 17 and 18. After step 1930 as described above with reference to FIG. 19 is performed, steps 2310 and 2320 may be performed.

In step 2310, the communication part of the diagnostic device receives an external feedback. For example, the communication part receives the external feedback using the user's interface of the diagnostic device. The external feedback may include the genetic characteristics of the user, the dietary habits of the user, the personal genetic characteristics of the user, and the like. As another example, the communication part may receive the external feedback from the server of FIG. 18.

The server 1840 may update data on the corresponding disease by using the received data. The server 1840 may have past and present liquid diagnostic information about patients having the corresponding disease, and may have data matching the liquid diagnostic information and actual symptoms of the patient. The server 1840 may predict disease progression for the liquid diagnostic information of the corresponding patient using the data of other patients. The server 1840 may transmit the predicted disease progression to the diagnostic device.

In step 2320, the processor of the diagnostic device adjusts or updates an algorithm for calculating a risk based on the external feedback. For example, if there is a user's family history for a specific disease, the algorithm may be adjusted to calculate a relatively high risk. As another example, the processor of the diagnostic device may reflect the disease progression received from the server 1840 to modify the algorithm.

FIG. 24 shows a method of controlling a medical device based on the risk according to an example.

The user 2410 may use a medical device 2440 to treat a specific disease. For example, the medical device 2440 may be a device for administering a drug to a user. As another example, the medical device 2440 may be a device that provides an electrical signal to the user 2410.

The user 2410 introduces a liquid into the diagnostic cartridge 2420. The diagnostic device 2430 calculates a risk for a specific disease using the diagnostic cartridge 2420. The diagnostic device 2430 controls an operation of the medical device 2440 based on the calculated risk. For example, when the risk is reduced, the medical device 2440 maybe controlled to reduce a drug or an electrical signal administered by the medical device 2440.

The device as described above may be embodied in a hardware component, a software component, and/or combinations of the hardware component and the software component. For example, the devices and components as described in the examples may be embodied using one or more general-purpose computers or special-purpose computers, such as, for example, a processor, a controller, an arithmetic logic unit (ALU), a digital signal processor, a microcomputer, a field programmable array (FPA), a programmable logic unit (PLU), a microprocessor, or any other device capable of executing and responding to instructions. The processing device may execute an operating system (OS) and one or more software applications running on the operating system. In addition, the processing device may access, store, manipulate, process, and generate data in response to the execution of the software. For the convenience of understanding, although the processing device may be described as one being used, those of ordinary skill in the art will appreciate that the processing device may comprise a plurality of processing elements and/or a plurality of types of processing elements. For example, the processing device may include a plurality of processors or one processor and one controller. In addition, other processing configurations such as a parallel processor are possible.

The software may include a computer program, code, instructions, or a combination of one or more of these, and may configure the processing device to operate as desired or may instruct the processing device independently or collectively. The software and/or data may be permanently or temporarily embodied in any type of machine, component, physical device, virtual equipment, computer storage medium or device, or signal wave being transmitted for the purpose of interpreting or providing instructions or data to the processing device, in order to be interpreted by the processing device or to provide instructions or data to the processing device. The software may be distributed over networked computer systems and stored or executed in a distributed manner. The software and data maybe stored on one or more computer-readable recording media.

The method according to the examples may be embodied in the form of program instructions, which may be executed through various computer means, and recorded on a computer-readable medium. The computer readable medium may include program instructions, data files, data structures, and the like, alone or in combination. The program instructions recorded on the media may be those specially designed and configured for the examples, or those that are well known and available to one of ordinary skill in the computer software art. Examples of computer-readable recording media include a magnetic media such as a hard disk, a floppy disks, and a magnetic tape, an optical media such as CD-ROM and DVD, magneto-optical media such as a floptical disk, and a hardware device specifically configured to store and execute program instructions, such as ROM, RAM, a flash memory, and the like. Examples of program instructions include not only machine language codes such as those generated by a compiler, but also high-level language codes that can be executed by a computer using an interpreter, or the like. The hardware device as described above may be configured to operate as one or more software modules to perform the operations of the examples, and vice versa.

Although the examples have been described by the limited examples and the drawings as described above, those of skill in the art will appreciate that various modifications and alterations may be made from the description above. For example, although the techniques as described herein is performed in a different order than the methods as described herein, and/or components such as the systems, structures, devices, circuits, and the like as described herein are joined or combined in a different form than the method as described herein, or replaced or substituted by other components or equivalents, appropriate results may be achieved.

### DESCRIPTION OF SYMBOLS

100: diagnostic cartridge

110: chip

120: biosensor

130: liquid receiving part

1. A diagnostic cartridge for diagnosing diabetes, comprising a nano wire-based biosensor formed based on an indium tin oxide microelectrode (ITO).

2. A diagnostic cartridge for detecting a target material included in a liquid, wherein the diagnostic cartridge comprises:
a top plate, in which a channel connecting an inlet and an outlet of the liquid is formed;
a chip coupled to the top plate to form the channel;
a biosensor adjacent to the channel; and
a circuit for supplying power to the biosensor, and
wherein when the target material binds to the biosensor, the resistance of the biosensor is changed.

3. The diagnostic cartridge according to claim 2, wherein the biosensor comprises:
a nano wire; and
a source electrode and a drain electrode, which are positioned at both ends of the nano wire and are connected to the circuit, respectively.

4. The diagnostic cartridge according to claim 3, wherein the nano wire is formed on a substrate of the biosensor based on an indium tin oxide microelectrode (ITO).

5. The diagnostic cartridge according to claim 4,
wherein the nano wire comprises an identification material capable of specifically binding to the target material,
wherein the identification material is fixed to the surface of the indium tin oxide (ITO).

6. The diagnostic cartridge according to claim 5, wherein the target material is an antigen, the identification material is an antibody, and the specific binding is an antigen-antibody response.

7. The diagnostic cartridge according to claim 6, wherein the antigen is a preset antigen for diagnosing diabetes.

8. The diagnostic cartridge according to claim 5, wherein when the target material and the identification material bind to each other, the resistance of the nano wire is changed by the binding.

9. The diagnostic cartridge according to claim 3, wherein the nano wire is plural.

10. The diagnostic cartridge according to claim 2, further comprising a liquid separation device for dividing the liquid into a plurality of channels, wherein the channel of the top plate is plural and the channels are connected to the liquid separation device, respectively.

11. The diagnostic cartridge according to claim 2, wherein the outlet comprises a pad capable of absorbing the liquid.

12. The diagnostic cartridge according to claim 2, wherein the biosensor is detachable from the chip.

13. A method of detecting a target material included in a liquid, wherein the method comprises the steps of:
supplying power to a diagnostic cartridge into which the liquid has been introduced;
measuring a current generated by the power; and
detecting the target material by determining whether the liquid includes the target material based on the measured current.

14. The method according to claim 13, wherein the diagnostic cartridge comprises:
a top plate, in which a channel connecting an inlet and an outlet of the liquid is formed;
a chip coupled to the top plate to form the channel;
a biosensor adjacent to the channel; and
a circuit for supplying power to the biosensor, and
wherein when the target material binds to the biosensor, the resistance of the biosensor is changed.

15. The method according to claim 14, wherein the biosensor comprises:
a nano wire; and
a source electrode and a drain electrode, which are positioned at both ends of the nano wire and are connected to the circuit, respectively.

16. The method according to claim 15, wherein the nano wire is formed on a substrate of the biosensor based on an indium tin oxide microelectrode (ITO).

17. The method according to claim 13, wherein the step of measuring the current generated by the power comprises the steps of:
measuring a plurality of currents generated by a plurality of nano wires of the biosensor; and
calculating the current based on the plurality of currents.

18. A computer-readable recording medium containing a program for performing the method according to any one of claims 13 to 17 claim 13.

19. An application for detecting a target material, which is stored in a data processing device, wherein the application controls the data processing device to perform the steps of:
supplying power to a diagnostic cartridge into which a liquid has been introduced;
measuring a current generated by the power; and
detecting the target material by determining whether the liquid includes the target material based on the measured current.

20. An apparatus for detecting a target material included in a liquid, wherein the apparatus comprises:
a memory in which a program for detecting the target material included in the liquid is recorded; and
a processor for executing the program, and
wherein the program comprises the steps of:
supplying power to a diagnostic cartridge into which the liquid has been introduced;
measuring a current generated by the power; and
detecting the target material by determining whether the liquid includes the target material based on the measured current.

21. A method of calculating a risk for a disease based on a target material included in a liquid, wherein the method comprises the steps of:
supplying power to a diagnostic cartridge into which the liquid has been introduced;
measuring a current generated by the power;
detecting the target material by determining whether the liquid includes the target material based on the measured current; and
calculating the risk for the disease using the target material and an algorithm.

22. An apparatus for calculating a risk for a disease based on a target material included in a liquid, wherein the apparatus comprises:
a memory in which a program for calculating the risk for the disease is recorded; and
a processor for executing the program, and
wherein the program comprises the steps of:
supplying power to a diagnostic cartridge into which the liquid has been introduced;
measuring a current generated by the power;
detecting the target material by determining whether the liquid includes the target material based on the measured current; and
calculating the risk for the disease using the target material and an algorithm.
